(19) United States
(12) Patent Application Publication
(10) Pub. No.: US 2025/0381104 A1
(43) Pub. Date: Dec. 18, 2025

(54) EMULSIFIED COSMETIC COMPOSITION CONTAINING HIGH CONTENT OF OIL

(71) Applicant: AMOREPACIFIC CORPORATION, Seoul (KR)

(72) Inventors: Young Suk Cho, Yongin-si (KR); Sung Shim Park, Yongin-si (KR); Jun Hyun Choi, Yongin-si (KR)

(73) Assignee: AMOREPACIFIC CORPORATION, Seoul (KR)

(21) Appl. No.: 19/238,926

(22) Filed: Jun. 16, 2025

(30) Foreign Application Priority Data
Jun. 18, 2024 (KR) 10-2024-0079221
May 19, 2025 (KR) 10-2025-0064497

(51) Int. Cl. A61K 8/06; A61K 8/81; A61Q 19/00

(52) U.S. Cl. CPC A61K 8/062; A61K 8/8147; A61Q 19/00; A61K 2800/10; A61K 2800/48

(57) ABSTRACT

An emulsified cosmetic composition is disclosed. More specifically, an emulsified cosmetic composition has an oil phase including a cationic polymer and an oil gelling agent, and a water phase including an anionic polymer. A method for producing the emulsified cosmetic composition is also disclosed.

### CROSS-REFERENCE TO RELATED APPLICATION

This application claims the benefit of priority under 35 U.S.C. § 119 (a) to Korean Patent Application No. 10-2024-0079221, filed on Jun. 18, 2024, and Korean Patent Application No. 10-2025-0064497, filed on May 19, 2025, the entire contents of which are hereby incorporated by reference.

### BACKGROUND

### (a) Technical Field

The present disclosure relates to an emulsified cosmetic composition having excellent stability at low temperatures, comprising an oil phase, a water phase, an ionic polymer and an oil gelling agent, and a method for producing the same.

### (b) Background Art

Emulsified cosmetic compositions have the advantage of having the moisture of skin/toner due to their low viscosity and providing a shielding effect and skin moisturizing feeling by maintaining functionality due to their high mixing amount of oil. Since these emulsified cosmetic compositions contain a high content of oil, they are produced in a way that a large amount of surfactant is mixed and a high-pressure emulsification process is performed, or in a way that a large amount of lecithin or polyglycerin-based surfactant is included and an emulsified two-phase form is formed.

Among them, the way that a large amount of surfactant is mixed and a high-pressure emulsification process is performed has the disadvantage that the contents would typically be required to prepare by applying high energy, and a high level of surfactant relative to the oil would need to be mixed.

The way that a large amount of lecithin or polyglycerin-based surfactant is included and an emulsified two-phase form is formed makes it difficult to maintain the initial appearance and quality because the emulsified particles coalesce at low temperatures. Specifically, since the emulsified particles adjacent to the gas phase have low fluidity at low temperatures, the phase inversion of the surfactant is induced, and the top layer of oil shows a phenomenon of coalescence, the initial appearance, usability and quality change.

In order to overcome the limitations of the prior art as described above, the present inventors have found that by mixing an ionic polymer capable of forming a coacervate and an oil gelling agent to form a thin film of an oil phase on top of a water phase, it is possible to improve low-temperature stability while maintaining emulsified particles, and have completed the present invention.

### SUMMARY OF THE DISCLOSURE

It is an object of the present invention to provide an emulsified cosmetic composition comprising a high content of oil, wherein the emulsified cosmetic composition has excellent stability at low temperatures by mixing an ionic polymer capable of forming a coacervate and an oil gelling agent.

It is another object of the present invention to provide a method for producing an emulsified cosmetic composition having excellent stability at low temperatures by stirring an oil phase including a cationic polymer and an oil gelling agent and a water phase including an anionic polymer.

To achieve the above object, one aspect of the present disclosure provides an emulsified cosmetic composition comprising an oil phase including a cationic polymer and an oil gelling agent; and a water phase including an anionic polymer.

In addition, one aspect of the present disclosure provides the emulsified cosmetic composition characterized in that the oil phase is located on top of the water phase and exists in a particle form.

In addition, one aspect of the present disclosure provides the emulsified cosmetic composition, wherein the cationic polymer includes a natural synthetic polymer.

In addition, one aspect of the present disclosure provides the emulsified cosmetic composition, wherein the oil gelling agent includes an ester-based oil and a urethane-based copolymer.

In addition, one aspect of the present disclosure provides the emulsified cosmetic composition, wherein the urethane-based copolymer includes one or more selected from the group consisting of Castor Oil/IPDI Copolymer, HDI/Trimethylol Hexyllactone Crosspolymer, Polyurethane-79, and combinations thereof.

In addition, one aspect of the present disclosure provides the emulsified cosmetic composition, wherein the anionic polymer includes a carbomer.

In addition, one aspect of the present disclosure provides the emulsified cosmetic composition, wherein the oil phase has a content of 5% by weight to 10% by weight based on the total weight of the cosmetic composition.

In addition, one aspect of the present disclosure provides the emulsified cosmetic composition, wherein the cationic polymer has a content of exceeding 0% by weight and 1% by weight or less based on the total weight of the cosmetic composition.

In addition, one aspect of the present disclosure provides the emulsified cosmetic composition, wherein the oil gelling agent has a content of exceeding 0% by weight and 1% by weight or less based on the total weight of the cosmetic composition.

In addition, one aspect of the present disclosure provides the emulsified cosmetic composition, wherein the anionic polymer has a content of exceeding 0% by weight and 0.5% by weight or less based on the total weight of the cosmetic composition.

In addition, one aspect of the present disclosure provides the emulsified cosmetic composition, wherein the oil phase further includes wax or fatty alcohol.

In addition, one aspect of the present disclosure provides the emulsified cosmetic composition, wherein the particle form has a size of 10 μm to 20 μm.

In addition, one aspect of the present disclosure provides the emulsified cosmetic composition, wherein the composition does not comprise a surfactant.

To achieve the above object, one aspect of the present disclosure provides a method for producing the emulsified cosmetic composition, comprising the steps of: (1) preparing an oil phase including a cationic polymer and an oil gelling agent; (2) preparing a water phase including an anionic polymer; and (3) stirring the oil phase and the water phase.

In addition, one aspect of the present disclosure provides the method for producing the emulsified cosmetic composition, wherein the oil gelling agent includes an ester-based oil and a urethane-based copolymer.

One aspect of the present disclosure improves stability at low temperatures by mixing an ionic polymer capable of forming a coacervate and an oil gelling agent into a cosmetic composition comprising a high content of oil, thereby allowing an emulsified cosmetic composition comprising a high content of oil to maintain its initial appearance, quality and usability until it reaches the final consumer even when distributed at low temperatures.

### BRIEF DESCRIPTION OF THE DRAWINGS

FIG. 1A is a schematic diagram of a two-phase cosmetic composition showing that, unlike an embodiment of the present invention, the oil phase coalesces and the oil phase and the water phase are separated into layers, and FIG. 1B is a schematic diagram of an emulsified cosmetic composition showing that, according to an embodiment of the present invention, the oil phase is positioned on top of the water phase and emulsified particles are maintained.

FIG. 2 is an optical microscope image (manufacturer: Nikon Instruments Korea Co., Ltd.; model name: Microscope 80i) (×200 magnification) showing the particle size of the emulsified cosmetic composition according to Example 1 of an embodiment of the present invention.

FIG. 3 is an optical microscope image (manufacturer: Nikon Instruments Korea Co., Ltd.; model name: Microscope 80i) (×200 magnification) showing the particle size of the emulsified cosmetic composition according to Comparative Example 2 of an embodiment of the present invention.

FIG. 4 shows a photograph taken of the state after Example 1 was stored for 4 weeks under CYC (−15° C. to 45° C.) temperature conditions.

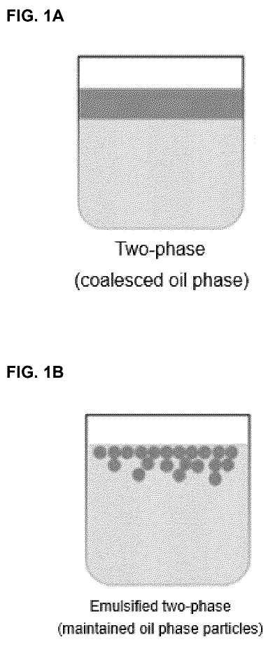

The left side of FIG. 5 shows a photograph taken of the state after Comparative Example 1 was stored for 1 week under CYC (−15° C. to 45° C.) temperature conditions, and the right side of FIG. 5 shows a photograph taken of the initial state of Comparative Example 1.

### DETAILED DESCRIPTION

Hereinafter, preferred embodiments of the present disclosure will be described in detail so that a person who has ordinary skill in the art to which the present disclosure pertains can easily practice the present disclosure.

Unless defined otherwise, all technical terms used in the present disclosure have the same meaning as commonly understood by those skilled in the relevant field of the present disclosure. In addition, those similar or equivalent to the preferred methods or samples described in the present disclosure are also included in the scope of the present disclosure.

Hereinafter, the composition of the emulsified cosmetic composition of the present disclosure will be examined more specifically.

### Emulsified Cosmetic Composition

The emulsified cosmetic composition according to one aspect of the present disclosure may comprise an oil phase and a water phase, and the oil phase may form a thin film on top of the water phase to maintain emulsified particles.

The oil phase may have a content of 5% by weight to 10% by weight based on the total weight of the cosmetic composition, and may be included in an amount of 6% by weight or more, 7% by weight or more, or 8% by weight or more; and
may be included in an amount of 9% by weight or less, 8% by weight or less, or 7% by weight or less.

In addition, it may be included in a range consisting of any one of the lower limits and any one of the upper limits as described above.

If the oil phase has a content of exceeding 10% by weight, the usability or stability may deteriorate due to excessive use of oil, and if it has a content of less than 5% by weight, due to the low mixing amount of oil, the functionality of the cosmetic cannot be sufficiently secured and the skin moisturizing feeling cannot be sufficiently provided.

The oil phase and the water phase of the emulsified cosmetic composition according to one aspect of the present disclosure may include substances having opposite potentials. Due to this, the oil phase and the water phase may exhibit opposite potentials, and a coacervate may be formed between the oil phase and the water phase due to charge interaction by the opposite potentials. Due to this, the oil phase forms a thin film on top of the water phase, and the oil phase exists in a particle form, so that the formulation of an emulsified cosmetic composition with excellent stability at low temperatures may be implemented.

In one aspect of the present disclosure, “coacervate” may mean that a cationic polymer and an anionic polymer are temporarily electrostatically bonded.

The oil phase of the emulsified cosmetic composition according to one aspect of the present disclosure may include a cationic polymer. The cationic polymer may be included without limitation as long as it is a polymer having positive electrical properties and can form a temporary coacervate with the anionic polymer.

The cationic polymer may include a natural synthetic polymer, and specifically, may include one or more selected from the group consisting of, but not limited to, chitosan, cationic cellulose, cationic guar, cationic polysaccharide, silicone-based polymers containing amino groups, polyquaternium, and combinations thereof, and preferably, the cationic polymer may include silicone-based polymers containing amino groups.

The silicone-based polymers containing the amino groups may include one or more selected from the group consisting of, but not limited to, bis-amodimethicone, amodimethicone, aminopropyl dimethicone, aminoethylaminopropyl dimethicone, amino bispropyl dimethicone, amodimethicone hydroxystearate, and combinations thereof, and preferably, may include aminopropyl dimethicone.

The cationic polymer may have a content of exceeding 0% by weight and 1% by weight or less based on the total weight of the cosmetic composition, and may be included in an amount of exceeding 0.1% by weight, exceeding 0.2% by weight, exceeding 0.3% by weight, exceeding 0.4% by weight, or exceeding 0.5% by weight; and
may be included in an amount of 0.9% by weight or less, 0.8% by weight or less, 0.7% by weight or less, 0.6% by weight or less, or 0.5% by weight or less.

In addition, it may be included in a range consisting of any one of the lower limits and any one of the upper limits as described above.

If the cationic polymer has a content of exceeding 1% by weight, there are problems in that the cationic polymers coalesce, resulting in a deterioration in the appearance and quality of the contents, and if it has a content of 0% by weight or less, the anionic polymer and coacervate cannot be sufficiently formed, making it impossible to maintain the stability of the contents.

The oil phase of the emulsified cosmetic composition according to one aspect of the present disclosure may include an oil gelling agent. The oil gelling agent may prevent the coalescence of emulsified particles at low temperatures, thereby increasing the stability of the contents and maintaining the initial appearance and quality.

The oil gelling agent may include an ester-based oil and a urethane-based copolymer. Specifically, it may include one or more selected from the group consisting of, but not limited to, Castor Oil/IPDI Copolymer and Caprylic/Capric Triglyceride marketed under the name Estogel M by PolymerExpert, Caprylic/Capric Triglyceride, Hydrogenated Poly(C6-20 Olefin) and HDI/Trimethylol Hexyllactone Crosspolymer marketed under the name Oilkemia 5S CC Polymer by Lubrizol Corporation, Caprylic/Capric Triglyceride and Polyurethane-79 marketed under the name Crystal Clear Wax by TKB Trading. LLC, and combinations thereof.

The ester-based oil may include, but is not limited to, Caprylic/Capric Triglyceride.

The urethane-based copolymer may include one or more selected from the group consisting of, but not limited to, Castor Oil/IPDI Copolymer, HDI/Trimethylol Hexyllactone Crosspolymer, Polyurethane-79, and combinations thereof.

The oil gelling agent may have a content of exceeding 0% by weight and 1% by weight or less based on the total weight of the cosmetic composition, and may be included in an amount of exceeding 0.1% by weight, exceeding 0.2% by weight, exceeding 0.3% by weight, exceeding 0.4% by weight, or exceeding 0.5% by weight; and
may be included in an amount of 0.9% by weight or less, 0.8% by weight or less, 0.7% by weight or less, 0.6% by weight or less, or 0.5% by weight or less.

In addition, it may be included in a range consisting of any one of the lower limits and any one of the upper limits as described above.

If the oil gelling agent has a content of exceeding 1% by weight, there is a disadvantage in that the hardness increases and the emulsified particles cannot be dispersed, and if it has a content of 0% by weight or less, the coacervate of the ionic polymers cannot be helped, making it impossible to maintain the stabilization of the contents.

The particle form in the oil phase of the emulsified cosmetic composition according to one aspect of the present disclosure may have a size of 10 μm to 20 μm, and may have a size of 11 μm or more, 12 μm or more, 13 μm or more, 14 μm or more, or 15 μm or more; and
may have a size of 19 μm or less, 18 μm or less, 17 μm or less, 16 μm or less, or 15 μm or less.

In addition, it may be included in a range consisting of any one of the lower limits and any one of the upper limits as described above.

If the particle size of the emulsified cosmetic composition is exceeding 20 μm, emulsified particles may not be sufficiently formed, and if it is less than 10 μm, there is a concern that the emulsified particles may coalesce at low temperatures, harming the stability of the product.

The oil phase of the emulsified cosmetic composition according to one aspect of the present disclosure may further include wax or fatty alcohol.

The wax may be a wax commonly used in cosmetic compositions, and specifically may include one or more selected from the group consisting of, but not limited to, ceresin wax, polyethylene wax, microcrystalline wax, paraffin wax, ozokerite, carnauba wax, candelilla wax, and combinations thereof.

The fatty alcohol may be a fatty alcohol commonly used in cosmetic compositions and may be solid at room temperature. Specifically, it may include one or more selected from the group consisting of, but not limited to, myristyl alcohol, cetyl alcohol, stearyl alcohol, and combinations thereof.

The water phase of the emulsified cosmetic composition according to one aspect of the present disclosure may include an anionic polymer. The anionic polymer may be included without limitation as long as it is a polymer having negative electrical properties and can form a temporary coacervate with the cationic polymer.

The anionic polymer may include, but is not limited to, a carbomer.

The anionic polymer may has a content of exceeding 0% by weight and 0.5% by weight or less based on the total weight of the cosmetic composition, and may be included in an amount of exceeding 0.01% by weight, exceeding 0.03% by weight, exceeding 0.05% by weight, exceeding 0.07% by weight, exceeding 0.09% by weight, exceeding 0.1% by weight, exceeding 0.2% by weight, or exceeding 0.3% by weight; and
may be included in an amount of 0.49% by weight or less, 0.47% by weight or less, 0.45% by weight or less, 0.43% by weight or less, 0.41% by weight or less, 0.4% by weight or less, 0.3% by weight or less, or 0.2% by weight or less.

In addition, it may be included in a range consisting of any one of the lower limits and any one of the upper limits as described above.

If the anionic polymer has a content of exceeding 0.5% by weight, there are problems in that the producing efficiency, and appearance and quality of the contents deteriorate, and if it has a content of 0% by weight or less, the cationic polymer and coacervate cannot be sufficiently formed, making it impossible to maintain the stability of the contents.

The water phase may further include polyol, etc. The polyol may be a polyol commonly used in cosmetic compositions, and specifically, may include one or more selected from the group consisting of, but not limited to, sorbitol, polyethylene glycol (PEG), polypropylene glycol (PPG), dipropylene glycol, propylene glycol, 1,3-butylene glycol, glycerin, propanediol, ethylhexanediol, 1,2-hexanediol, PEG/PPG/polybutylene glycol-8/5/3 glycerin, ethylhexylglycerin, pentylene glycol, and combinations thereof.

The emulsified cosmetic composition according to one aspect of the present disclosure may maintain the stability of the formulation even without containing a surfactant. In the past, surfactants were used as an essential component in emulsified cosmetic compositions comprising a high content of oil to stabilize the formulation. However, surfactants sometimes cause a sticky feeling in cosmetic compositions or cause skin irritation. According to the one aspect of the present disclosure, by mixing an ionic polymer capable of forming a coacervate and an oil gelling agent, the formulation may be stabilized without using a surfactant.

The emulsified cosmetic composition according to one aspect of the present disclosure may further comprise one or more selected from the group consisting of all kinds of ingredients that can be used in conventional cosmetics, whitening effect ingredients, anti-wrinkle ingredients, fragrances, preservatives, antioxidants, anti-discoloration agents, and combinations thereof, within a range that does not impede the purpose of the present disclosure.

### Method for Producing an Emulsified Cosmetic Composition

The method for producing the emulsified cosmetic composition according to one aspect of the present disclosure may comprise the steps of: (1) preparing an oil phase including a cationic polymer and an oil gelling agent; (2) preparing a water phase including an anionic polymer; and (3) stirring the oil phase and the water phase.

Since the cationic polymer, oil gelling agent, and anionic polymer used in the method for producing the emulsified cosmetic composition according to one aspect of the present disclosure have already been described above, the specific description will be omitted below.

The method for producing the emulsified cosmetic composition according to one aspect of the present disclosure may comprise the step of (1) preparing an oil phase including a cationic polymer and an oil gelling agent.

In the step of preparing the oil phase, a method may be used, such as preparing an oil phase by completely dissolving the components using a paddle mixer at 80° C. to 110° C. Since the oil phase includes an oil gelling agent, a dissolution process may be involved.

The method for producing the emulsified cosmetic composition according to one aspect of the present disclosure may comprise the step of (2) preparing a water phase including an anionic polymer.

In the step of preparing the water phase, a method may be used, such as preparing a water phase by stirring the components using a homomixer at a high temperature (about 75° C.). The water phase requires dispersion of a thickener, and requires heating conditions because it applies a high-temperature emulsification process.

The method for producing the emulsified cosmetic composition according to one aspect of the present disclosure may comprise the step of (3) stirring the oil phase and the water phase.

In the step of stirring the oil phase and the water phase, a conventional method of stirring the oil phase and the water phase may be used, such as slowly adding the oil phase to the water phase while stirring.

Hereinafter, the contents of the present disclosure will be explained more specifically through examples and test examples. It will be apparent to those skilled in the art that the following examples and test examples are only illustrated to more specifically explain the present disclosure and the scope of the present disclosure is not to be construed as being limited to these examples and test examples.

### Examples 1 to 3 and Comparative Examples 1 to 5

Emulsified cosmetic compositions of Examples 1 to 3 and Comparative Examples 1 to 5 were produced according to the compositions described in Tables 1 and 2 below.

Specifically, the components included in the oil phase in the reactor in the composition ratios shown in Tables 1 and 2 were sufficiently heated and completely dissolved at 110° C. using a paddle mixer (manufacturer: Global lab; model name: SS-20) to prepare the oil phase. Separately, the components included in the water phase in the composition ratios shown in Tables 1 and 2 were heated to 75° C., and then stirred at 9000 rpm for 3 minutes using a homomixer (manufacturer: Primix, model name: Mark2 2.5) to prepare the water phase.

The prepared oil phase was slowly added to the prepared water phase while stirring at a low speed of 3000 rpm for 3 minutes to produce emulsified cosmetic compositions.

In the case of Comparative Example 2, hydrogenated lecithin was also added as a surfactant while adding the prepared oil phase to the prepared water phase.

To evaluate stability, trace amounts of oil-soluble pigments (types: MALACHITE EXTRACT, LITHOSPERMUM ERYTHRORHIZON ROOT EXTRACT) were added to Examples 1 to 3 and Comparative Examples 1 to 5.

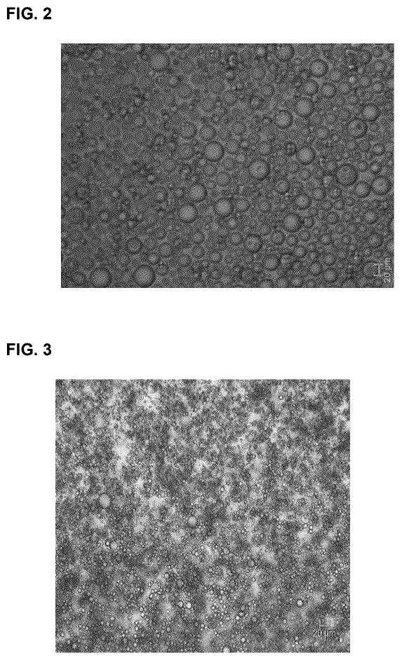

TABLE 1
(Unit: % by weight)

| Ingredients/Types | | Example 1 | Example 2 | Example 3 |
|---|---|---|---|---|
| Oil | Squalane | 2 | 2 | 2 |
| phase | Isononyl isononanoate | 4 | 4 | 4 |
| | Oilkemia 5S CC polymer | 0.5 | — | — |
| | Crystal clear wax | — | 0.5 | — |
| | EstoGel M | — | — | 0.5 |
| | Oleogy LEOT | — | — | — |
| | HDXP | — | — | — |
| | Aminopropyl dimethicone | 0.1 | 0.1 | 0.1 |
| Surfac- | Hydrogenated lecithin | — | — | — |
| tant | | | | |
| Water | Carbopol 981 | 0.05 | 0.05 | 0.05 |
| phase | SEPINOV WEO | — | — | — |
| | Purified water (D.I. water) | Up to 100 | Up to 100 | Up to 100 |
| | Glycerine | 3 | 3 | 3 |
| | Propandiol | 7 | 7 | 7 |
| | 1,2-Hexandiol | 1.8 | 1.8 | 1.8 |
| | Ethylhexylglycerin | 0.1 | 0.1 | 0.1 |

| | | | |
|---|---|---|---|
| Oil-soluble pigment | trace | trace | trace |

[FIG. 2]

(Unit: % by weight)

| | | Comparative | Comparative | Comparative | Comparative | Comparative |
|---|---|---|---|---|---|---|
| Ingredients/Types | | Example 1 | Example 2 | Example 3 | Example 4 | Example 5 |
| Oil phase | Squalane | 2 | 2 | 2 | 2 | 2 |
| | Isononyl isononanoate | 4 | 4 | 4 | 4 | 4 |
| | Oilkemia 5S CC polymer | — | — | — | — | 0.5 |
| | Crystal clear wax | — | — | — | — | — |
| | EstoGel M | — | — | — | — | — |
| | Oleogy LEOT | — | — | 0.5 | — | — |
| | HDXP | — | — | — | 0.5 | — |
| | Aminopropyl | 0.1 | — | 0.1 | 0.1 | 0.1 |
| | dimethicone | | | | | |
| Surfactant | Hydrogenated lecithin | — | 0.5 | — | — | — |
| Water | Carbopol 981 | 0.05 | — | 0.05 | 0.05 | — |
| phase | SEPINOV WEO | — | — | — | — | 0.1 |
| | Purified water | Up to 100 | Up to 100 | Up to 100 | Up to 100 | Up to 100 |
| | (D.I. water) | | | | | |
| | Glycerine | 3 | 3 | 3 | 3 | 3 |
| | Propandiol | 7 | 7 | 7 | 7 | 7 |
| | 1,2-Hexandiol | 1.8 | 1.8 | 1.8 | 1.8 | 1.8 |
| | Ethylhexylglycerin | 0.1 | 0.1 | 0.1 | 0.1 | 0.1 |

| | | | | | |
|---|---|---|---|---|---|
| Oil-soluble pigment | trace | trace | trace | trace | trace |

* Oilkemia 5S CC polymer: Caprylic/Capric Triglyceride, Hydrogenated Poly(C6-20 Olefin) and HDI/Trimethylol Hexyllactone Crosspolymer
* Crystal clear wax: Caprylic/Capric Triglyceride and Polyurethane-79
* EstoGel M: Castor Oil/IPDI Copolymer and Caprylic/Capric Triglyceride
* Oleogy LEOT: Octyldodecanol, Dibutyl Ethylhexanoyl Glutamide and Dibutyl Lauroyl Glutamide
* HDXP: Dextrin palmitate
* Carbopol 981: Carbomer
* SEPINOV WEO: Hydroxyethyl Acrylate/Sodium Acryloyldimethyl Taurate Copolymer

### [Test Example 1] pH Evaluation

The pH of the emulsified cosmetic compositions of Examples 1 to 3 and Comparative Examples 1 to 5 was measured and summarized in Table 3.

TABLE 3

| | | | | Comparative | Comparative | Comparative | Comparative | Comparative |
|---|---|---|---|---|---|---|---|---|
| | Example 1 | Example 2 | Example 3 | Example 1 | Example 2 | Example 3 | Example 4 | Example 5 |
| pH | 5.1 | 5.3 | 5.0 | 5.2 | 5.6 | 5.1 | 5.2 | 6.0 |

Cosmetics can be distributed only when the pH is measured as 3.0 to 9.0, and all of Examples 1 to 3 and Comparative Examples 1 to 5 were measured to satisfy the above range.

### [Test Example 2] Evaluation of Particle Sizes

The particle sizes of the emulsified cosmetic compositions according to Example 1 and Comparative Examples 1 and 2 were measured using an optical microscope (manufacturer: Nikon Instruments Korea Co., Ltd.; model name: Microscope 80i; ×200 magnification), and Example 1 and Comparative Examples 2 are shown in FIGS. 2 and 3.

The particle sizes of Example 1 and Comparative Example 1 were measured at the level of 10 μm to 20 μm, and those of Comparative Example 2 were measured at the level of 1 μm to 10 μm. It could be confirmed that Comparative Example 2 included a surfactant, but did not include a cationic polymer, an anionic polymer and an oil gelling agent, and thus the particle size was formed to be small.

### [Test Example 3] Evaluation of Appearance and Stability of Emulsified Cosmetic Compositions

In order to confirm the appearance of the emulsified cosmetic compositions, the emulsified cosmetic compositions according to Examples 1 to 3 and Comparative Examples 1 to 5 were placed in transparent plastic containers and their appearances were observed, which are shown in Table 4.

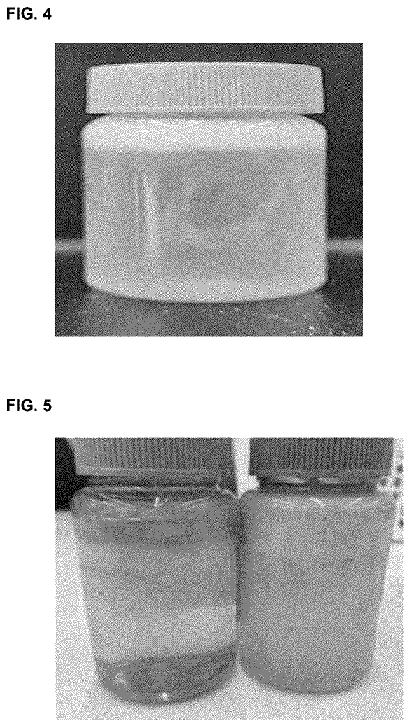

In order to confirm the stability of the emulsified cosmetic compositions, the emulsified cosmetic compositions according to Examples 1 to 3 and Comparative Examples 1 to 5 were placed in transparent plastic containers and stored for 4 weeks under CYC (−15° C. to 45° C.) temperature conditions, and the degree of separation over time was observed, which is shown in Table 4. In addition, a photograph taken of the state after 4 weeks of Example 1 is shown in FIG. 4, and a photograph taken of the state after 1 week of Comparative Example 1 is shown on the left side of FIG. 5.

TABLE 4

| | | | | Comparative | Comparative | Comparative | Comparative | Comparative |
|---|---|---|---|---|---|---|---|---|
| | Example 1 | Example 2 | Example 3 | Example 1 | Example 2 | Example 3 | Example 4 | Example 5 |
| Appearance | Emulsified | Emulsified | Emulsified | Emulsified | Emulsified | Emulsified | Emulsified | Emulsified |
| | two-phase | two-phase | two-phase | two-phase | two-phase | single-phase | single-phase | two-phase |
| Stability | Good | Good | Good | Separated | Separated | Separated | Separated | Separated |

With regard to appearance, Examples 1 to 3, which comprise an oil gelling agent including a cationic polymer, an ester-based oil and a urethane copolymer in the oil phase and comprise a carbomer in the water phase, exhibited an emulsified two-phase; Comparative Examples 3 and 4, which do use ingredients other than the oil gelling agent, exhibited an emulsified single-phase; and Comparative Examples 1 and 2, which do not use an oil gelling agent, and Comparative Example 5, which uses the oil gelling agent, exhibited an emulsified two-phase.

With regard to stability, only Examples 1 to 3 including the ingredients had good stability, and Comparative Examples 1 to 5 not including all of the ingredients showed separation. For example, it could be confirmed that in Example 1, which includes all of a cationic polymer, an anionic polymer and an oil gelling agent, no separation occurred under all temperature (CYC, −15° C., 30° C., 37° C., 45° C.) conditions even after 4 weeks and no color change was observed, and thus, the emulsified cosmetic compositions according to one aspect of the present disclosure stably maintained its initial appearance and quality even at low temperatures.

However, it was confirmed that in the case of Comparative Example 1, which includes a cationic polymer and an anionic polymer but does not include an oil gelling agent, although separation did not occur initially, separation occurred while undergoing a low-temperature state, and thus, the initial appearance and quality could not be maintained after 1 week. It was also confirmed that in Comparative Example 2, which includes a surfactant but does not include a cationic polymer, an anionic polymer and an oil gelling agent, separation occurred at low temperatures, and thus, the initial appearance and quality could not be maintained.

What is claimed is:

1. An emulsified cosmetic composition, comprising:
an oil phase including a cationic polymer and an oil gelling agent; and
a water phase including an anionic polymer.

2. The emulsified cosmetic composition according to claim 1, wherein the oil phase is located on top of the water phase and exists in a particle form.

3. The emulsified cosmetic composition according to claim 1, wherein the cationic polymer includes a natural synthetic polymer.

4. The emulsified cosmetic composition according to claim 1, wherein the oil gelling agent includes an ester-based oil and a urethane-based copolymer.

5. The emulsified cosmetic composition according to claim 4, wherein the urethane-based copolymer includes one or more selected from the group consisting of Castor Oil/IPDI Copolymer, HDI/Trimethylol Hexyllactone Crosspolymer, Polyurethane-79, and combinations thereof.

6. The emulsified cosmetic composition according to claim 1, wherein the anionic polymer includes a carbomer.

7. The emulsified cosmetic composition according to claim 1, wherein the oil phase has a content of 5% by weight to 10% by weight based on the total weight of the cosmetic composition.

8. The emulsified cosmetic composition according to claim 1, wherein the cationic polymer has a content of exceeding 0% by weight and 1% by weight or less based on the total weight of the cosmetic composition.

9. The emulsified cosmetic composition according to claim 1, wherein the oil gelling agent has a content of exceeding 0% by weight and 1% by weight or less based on the total weight of the cosmetic composition.

10. The emulsified cosmetic composition according to claim 1, wherein the anionic polymer has a content of exceeding 0% by weight and 0.5% by weight or less based on the total weight of the cosmetic composition.

11. The emulsified cosmetic composition according to claim 1, wherein the oil phase further includes wax or fatty alcohol.

12. The emulsified cosmetic composition according to claim 2, wherein the particle form has a size of 10 μm to 20 μm.

13. The emulsified cosmetic composition according to claim 1, wherein the composition does not comprise a surfactant.

14. A method for producing the emulsified cosmetic composition according to claim 1, comprising the steps of:
(1) preparing an oil phase including a cationic polymer and an oil gelling agent;
(2) preparing a water phase including an anionic polymer; and
(3) stirring the oil phase and the water phase.

15. The method for producing the emulsified cosmetic composition according to claim 14, wherein the oil gelling agent includes an ester-based oil and a urethane-based copolymer.
